(19) United States
(12) Patent Application Publication
(10) Pub. No.: US 2021/0128569 A1
(43) Pub. Date: May 6, 2021

(54) ORODISPERSIBLE FORMULATION OF VARDENAFIL

(71) Applicant: Alpex Pharma S.A., Mezzovico (CH)

(72) Inventors: Federico STROPPOLO, Aldesago (CH); Gabriele GRANATA, Leggiuno (VA) (IT)

(73) Assignee: Alpex Pharma S.A., Mezzovico (CH)

(21) Appl. No.: 17/075,907

(22) Filed: Oct. 21, 2020

(30) Foreign Application Priority Data
Nov. 5, 2019 (IT) 102019000020350

(51) Int. Cl. A61K 31/53; A61K 9/20

(52) U.S. Cl. CPC A61K 31/53; A61K 9/2018; A61K 9/2031

(57) ABSTRACT

The present invention includes an orodispersible polyalcohol-free formulation containing vardenafil or a pharmaceutically acceptable salt thereof as active ingredient, as well as filler, a buffering agent, a lubricant, a sweetener, a flavoring agent and, optionally, further pharmaceutically acceptable excipients.

### CROSS-REFERENCE TO RELATED APPLICATIONS

This application claims the benefit of priority from Italian Patent Application No. 102019000020350 filed on Nov. 5, 2019, the contents of which are incorporated herein by reference.

### FIELD OF THE INVENTION

The present invention relates to an orodispersible formulation (ODT) and in particular to an orodispersible formulation containing vardenafil or a pharmaceutically acceptable salt thereof as active ingredient.

### BACKGROUND OF THE INVENTION

Vardenafil, having as chemical name 2-[2-ethoxy-5-(4-ethylpiperazin-1-il)sulfonylphenyl]-5-methyl-7-propyl-1H-imidazo[5,1-f][1,2,4]triazin-4-one, is a known active ingredient as PDES inhibitor used for the treatment of erectile dysfunctions.

ODT formulations of vardenafil are already known in literature and available on the market. In particular, an ODT formulation containing vardenafil hydrochloride is commercialized under the trade names Staxyn® and Levitra® Soft.

This formulation is disclosed in the U.S. Pat. No. 8,613,950 (Bayer) and is particularly advantageous since, further to the known administration advantages linked to the ODT formulations (such as ease of administration also for patients with swallowing difficulties, greater compliance, etc.), it also demonstrates an increased bioavailability of the active ingredient and a plateau-like plasma concentration profile.

In order to obtain these advantageous characteristics, it is however mandatory, as reported in U.S. Pat. No. 8,613,950, that the formulation contains at least one polyalcohol, in particular mannitol, sorbitol or mixtures thereof. As a matter of fact, the commercially available formulation contains a mixture of mannitol and sorbitol.

We have now surprisingly found that the same advantageous characteristics of bioavailability and plasmatic profile of vardenafil of ODT formulations disclosed in U.S. Pat. No. 8,613,950 can be obtained with a polyalcohol-free ODT formulation.

### SUMMARY OF THE INVENTION

An object of the present invention is therefore an orodispersible formulation (ODT), polyalcohol-free and that contains vardenafil or a pharmaceutically acceptable salt thereof as active ingredient, comprising at least one filler, at least one buffering agent, at least one lubricant, at least one sweetener, at least one flavoring agent and optionally further pharmaceutically acceptable excipients.

### DETAILED DESCRIPTION OF THE INVENTION

The formulations of the present invention are here defined as “orodispersible” according to the European Pharmacopoeia (disintegration time <3 minutes). On the other hand, FDA guidelines report as definition of “orally disintegrating tablet” (ODT) a solid oral formulation having a disintegration time of about 30 seconds or less. The formulations of the present invention can be indicated either as orodispersible formulations or as ODT formulations.

Several salts of vardenafil are known in literature either with inorganic acids (for example hydrochloride) or with organic acids (for example citrate). The formulations of the present invention preferably contain vardenafil hydrochloride, still more preferably vardenafil monohydrochloride, generally used in the trihydrate form.

Vardenafil or a pharmaceutically acceptable salt thereof is present in an amount ranging from 2% to 20% by weight, preferably from 5% to 10%.

The quantity by weight of the active ingredient generally corresponds to a dose of vardenafil free base of 10 mg per dosage unit (tablet) in many aspects of the invention.

The feature of the formulations of the present invention is that of being polyalcohol-free.

In this application, the term “polyalcohol” is used in its definition commonly known to those skilled in the art, i.e. referring to compounds of general formula HOCH₂(CHOH)ₙCH₂OH derived from sugars. Among the most common polyalcohols used as pharmaceutical excipients, mannitol, sorbitol, inositol, xylitol, maltitol and lactitol can be cited.

The formulation of the present invention contains at least one filler, at least one buffering agent, at least one lubricant, at least one sweetener, at least one flavoring agent and optionally further pharmaceutically acceptable excipients.

Fillers are excipients used for increasing the weight of the formulation in order to improve the workability thereof. They are generally present in amounts greater than 50% by weight. The filler preferably used in the formulation of the present invention is trisodium citrate, but other common fillers can be used such as monosodium phosphate, disodium phosphate, trisodium phosphate, monosodium citrate and disodium citrate.

Trisodium citrate is generally used in a weight ratio relative to the active ingredient ranging from 1:10 to 1:3 (ratio active ingredient:trisodium citrate).

Buffering agents are generally organic or inorganic acids paired with a salt thereof that allow to keep the pH within a certain range. Citric acid and its salts are among the most commonly used buffering agents in pharmaceutical technology. In the present invention, the mixture citric acid/trisodium citrate is preferably used.

Trisodium citrate is used in large excess with respect to citric acid, thus exercising both the function of filler and that of basic component of the buffering agent.

Preferably, the weight ratio between citric acid and trisodium citrate ranges from 1:3 to 1:15 and in some preferable embodiments is 1:7.

Lubricants are excipients used for improving the workability of formulations since they reduce the friction of the powder or of the granulate in the tablet press machines and thus prevent their adhesion to the punches and to the mold walls.

Common lubricants used in pharmaceutical technology are stearic acid and magnesium or calcium stearate, talc, paraffine, sodium lauryl sulfate, PEG having different molecular weight (PEG 300, 400, 600, 6000, 8000, etc.) and sodium benzoate.

In the present invention soluble lubricants are preferably used such as sodium lauryl sulfate, PEG and sodium benzoate. Still more preferably sodium benzoate, PEG 6000 or mixtures thereof are used. One particularly preferred embodiment is a mixture of sodium benzoate and PEG 6000.

In the formulation of the present invention, the preferred amount of PEG 6000 is 3-20% by weight and the amount of sodium benzoate is 5-15% by weight.

In the formulation of the present invention there can be present also small amounts of sweetener and flavoring agent to improve the organoleptic characteristics.

Preferred sweeteners are artificial sweeteners such as potassium acesulfame, aspartame, cyclamate, saccharin, sucralose, preferably sucralose. The amount of artificial sweetener is preferably ranging from 1% to 5% by weight of the formulation.

Preferred flavoring agents are mint flavor, anise flavor, licorice flavor, vanilla flavor and mixtures thereof. Flavoring agents are used in an amount preferably ranging from 3% to 8% by weight of the formulation. The preferred flavoring agent is mint flavor in an amount ranging from 4% to 5% by weight.

In the formulation of the present invention there can be added also further excipients among those commonly used in the preparation of orodispersible formulations. As a non-limitative example, such further excipients can be colorants, anti-adhesives, disintegrants, etc.

The formulations of the present invention are prepared according to conventional methods such as dry mixing, wet granulation, dry granulation, etc.

The formulations of the present invention are solid oral formulations, preferably tablets, that disintegrate quickly in the mouth and ensure a bioavailability of the active ingredient equivalent to that of the reference product on the market (Staxyn®/Levitra®).

In order to better illustrate the present invention, without limiting it, the following examples are now provided.

### Example 1

A formulation having the following composition was prepared:

| | Composition per | | Quantities for a |
|---|---|---|---|
| | tablet | | batch of 14 Kg |
| Components | mg | % | Kg |
| Vardenafil hydrochloride | 11.85 | 8.46 | 1.185 |
| trihydrate (equivalent to | | | |
| 10 mg of vardenafil) | | | |
| Trisodium citrate | 87.15 | 62.25 | 8.715 |
| anhydrous | | | |
| Citric acid | 13.00 | 9.29 | 1.300 |
| PEG 6000 | 8.5 | 6.07 | 0.850 |
| Sodium benzoate in | 8.5 | 6.07 | 0.850 |
| powder | | | |
| Sucralose | 4.00 | 2.86 | 0.400 |
| Peppermint aroma | 7.00 | 5.00 | 0.700 |
| TOTAL | 140 | 100 | 14 |

Trisodium citrate anhydrous, citric acid, PEG 6000, aroma and sucralose were placed in a cubic mixer and the mixture was mixed for about 10 minutes. Then, the vardenafil hydrochloride trihydrate and sodium benzoate were added, and the mixture was mixed for further 10 minutes. The resulting mixture was then compressed in a rotary tablet press machine equipped with 7 mm diameter punches in order to prepare tablets weighing 140 mg. The tablets were chemically and physically analyzed and then packed individually in an aluminum/aluminum blister.

### Example 2

A batch having the same composition as the batch described in Example 1 was prepared according to the following procedure.

Trisodium citrate anhydrous, citric acid, PEG 6000, aroma and sucralose were placed in a fluid bed granulator. The mixture was granulated by using around 1000 g of purified water and dried until obtaining a content of residual moisture below 5%. The obtained granulate was then placed in a cubic mixer and vardenafil hydrochloride trihydrate and sodium benzoate were added. The mixture was mixed for 10 minutes. The resulting mixture was then compressed in a rotary tablet press machine equipped with 7 mm diameter punches in order to prepare tablets weighing 140 mg. The tablets were chemically and physically analyzed and then packed individually in an aluminum/aluminum blister.

### Example 3

A batch having the same composition as the batch described in Example 1 was prepared according to the following procedure.

Trisodium citrate anhydrous, citric acid, PEG 6000, aroma and sucralose were placed in a cubic mixer and the mixture was mixed for about 10 minutes. The mixture was then compacted into blocks of about 5 g each by using a rotary tablet press machine. The blocks were crushed by means of an oscillating granulator equipped with a net having a 1 mm diameter clear span and loaded into a cubic mixer. Vardenafil hydrochloride trihydrate and sodium benzoate were added, and the mixture was mixed for 10 minutes. The resulting mixture was then compressed in a rotary tablet press machine equipped with 7 mm diameter punches in order to prepare tablets weighing 140 mg. The tablets were chemically and physically analyzed and then packed individually in an aluminum/aluminum blister.

### Example 4

Operating according to a procedure similar to that described in Example 1, a 20 Kg batch was prepared and 200 mg tablets having the following composition:

| | Composition per | | Quantities for a |
|---|---|---|---|
| | tablet | | batch of 20 Kg |
| Components | mg | % | Kg |
| Vardenafil hydrochloride | 11.85 | 5.925 | 1.185 |
| trihydrate (equivalent to | | | |
| 10 mg of vardenafil) | | | |
| Trisodium citrate | 110.00 | 55.000 | 11.000 |
| anhydrous | | | |
| Citric acid | 25.15 | 12.575 | 2.515 |
| PEG 6000 | 30.0 | 15.000 | 3.000 |
| Sodium benzoate in | 10.0 | 5.000 | 1.000 |
| powder | | | |
| Sucralose | 5.00 | 2.500 | 0.500 |
| Peppermint aroma | 8.00 | 4.000 | 0.800 |
| TOTAL | 200 | 100 | 20 |

### Example 5

Operating according to a procedure similar to that described in Example 2, a 20 Kg batch was prepared and 200 mg tablets having the composition disclosed in Example 4.

### Example 6

Operating according to a procedure similar to that described in Example 2 but compacting the mixture with a roller compactor, a 20 Kg batch was prepared and 200 mg tablets having the composition disclosed in Example 4.

### Example 7

The physical characteristics of the tablets according to the present invention were compared with those of the commercialized tablets under the trade name Staxyn®, i.e. the tablets disclosed in U.S. Pat. No. 8,613,950 (reference product). The values of the measured and compared parameters are reported in the following table:

| | | | | | | | Reference |
|---|---|---|---|---|---|---|---|
| Parameters | Ex. 1 | Ex. 2 | Ex. 3 | Ex. 4 | Ex. 5 | Ex. 6 | product |
| Diameter- mm | 7 | 7 | 7 | 10 | 10 | 10 | 9 |
| Weight - mg | 140 | 140 | 140 | 200 | 200 | 200 | 180 |
| Hardness - KP | 2 | 3 | 2 | 2 | 3 | 2 | 2.6 |
| Crumbliness- % | 1 | 1 | 1 | 1 | 1 | 1 | 1 |
| Disintegration | 30″ | 30″ | 30″ | 30″ | 30″ | 30″ | 30″ |
| time- seconds | | | | | | | |

The tablets of the invention display physicochemical characteristics similar to those of the reference product. Moreover, the tablets of the invention display a bioavailability profile of the active ingredient equivalent to that of the reference product.

1. An orally disintegrating polyalcohol-free formulation, comprising vardenafil or a pharmaceutically acceptable salt thereof, a filler, a buffering agent, a lubricant, a sweetener, a flavoring agent and, optionally further pharmaceutically acceptable excipients.

2. The formulation of claim 1, wherein the active ingredient is vardenafil hydrochloride.

3. The formulation of claim 2, wherein the vardenafil hydrochloride is vardenafil monohydrochloride trihydrate.

4. The formulation of claim 1, wherein the vardenafil or pharmaceutically acceptable salt thereof is present in an amount of from 2% to 20% by weight.

5. The formulation of claim 4, wherein the vardenafil or pharmaceutically acceptable salt thereof is present in an amount of from 5% to 10% by weight.

6. The formulation of claim 1, wherein the filler is trisodium citrate, optionally present in a weight ratio relative to the vardenafil or a pharmaceutically acceptable salt thereof of from 1:10 to 1:3.

7. The formulation of claim 1, wherein the buffering agent is a citric acid/trisodium citrate mixture used in a weight ratio of citric acid:trisodium citrate from 1:3 to 1:15.

8. The formulation of claim 1, wherein the lubricant is selected from the group consisting of sodium benzoate, PEG 6000 and mixtures thereof.

9. The formulation of claim 8, wherein the lubricant is a mixture of sodium benzoate and PEG 6000

10. The formulation of claim 9, wherein the amount of PEG 6000 is 3-20% by weight of the formulation and the amount of sodium benzoate is 5-15% by weight of the formulation.

11. The formulation of claim 1, wherein the sweetener is sucralose, optionally in an amount ranging from 1% to 5% by weight of the formulation.

12. The formulation of claim 1, wherein the flavoring agent is selected from the group consisting of mint flavor, anise flavor, licorice flavor, vanilla flavor and mixtures thereof, optionally present in an amount ranging from 3% to 8% by weight of the formulation.

13. The formulation of claim 12, wherein the flavoring agent is mint flavor.

14. The formulation of claim 12, wherein the flavoring agent is present in an amount ranging from 4% to 5% by weight.

15. A formulation of claim 1 in the form of a tablet.
